(19) United States
(12) Patent Application Publication
(10) Pub. No.: US 2005/0209576 A1
(43) Pub. Date: Sep. 22, 2005

(54) Disposable absorbent articles contained in package having window

(76) Inventor: Dennis Osamu Hirotsu, Higashinada-ku (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/130,674

(22) Filed: May 17, 2005

Related U.S. Application Data
(63) Continuation of application No. 10/719,321, filed on Nov. 21, 2003.
Application No. 10/719,321 is a continuation of application No. PCT/US02/16048, filed on May 21, 2002.
(60) Provisional application No. 60/294,515, filed on May 30, 2001.

(51) Int. Cl. A61F 13/15

(52) U.S. Cl. 604/385.02

(57) ABSTRACT

A disposable absorbent product having a package with at least one window and an outer surface and a plurality of disposable absorbent articles contained in the package. The plurality of disposable absorbent articles include one type of absorbent article wherein each absorbent article is identified by an indication means disposed on the respective absorbent articles. The type of absorbent article has a predetermined absorbency which is identified by an indication means disposed on the respective absorbent articles. The indication means has a color which indicates a predetermined absorbency. The indication means of the absorbent articles can be seen through the at least one window.

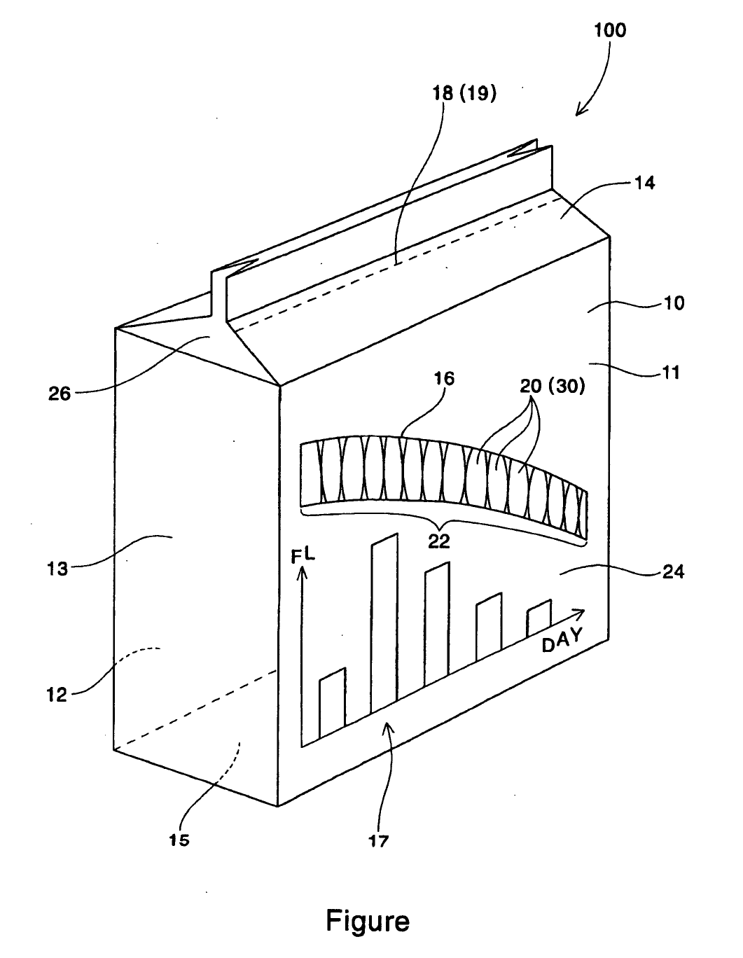

### CROSS REFERENCE TO RELATED APPLICATION

This application is a continuation of U.S. application Ser. No. 10/719,321, filed Nov. 21, 2003, pending which is a continuation of prior copending International Application No. PCT/US02/16048, filed May 21, 2002, designating the U.S.; which claims benefit of Provisional Application Ser. No. 60/294,515 filed May 30, 2001.

### FIELD OF THE INVENTION

The present invention relates to disposable absorbent products, and more particularly to a disposable absorbent product which contains disposable absorbent articles in a package having a window.

### BACKGROUND OF THE INVENTION

Disposable absorbent articles such as sanitary napkins, tampons, interlabial products, incontinence devices, pantiliners, diapers, incontinent briefs, incontinent undergarments, and training pants are typically worn in the crotch region of an undergarment to receive and contain body fluids. Such disposable absorbent articles function both to contain discharged materials and to isolate these materials from the body of the wearer and from the wearer's garments and bed clothing. Disposable absorbent articles having many different basic designs are known in the art. For example, sanitary napkins are one kind of absorbent article worn by women in a pair of panties that is normally positioned between the wearer's legs, adjacent to the perineal area of the body.

In general, the absorbent capacity of a disposable absorbent article necessary to provide containment of bodily fluids without leaking is quite different between the daytime use and the overnight use. Since the wearing time is usually longer in the nighttime as compared to the daytime, the quantity of bodily fluids discharged during the overnight use is typically greater than the quantity of bodily fluids discharged during the daytime use. In addition, in an example of catamenial protection, since the level of menstrual fluids discharged changes throughout the menstruation period, the use of feminine hygiene articles having different absorbent capacities is preferred by users. Even though the absorbent capacity requirements are quite different, disposable absorbent articles having one level of absorbent capacity are typically contained in a single package and sold in the market. As a result, a user who wants to use absorbent articles having different absorbent capacities needs to purchase a plurality of disposable absorbent products to meet the needs for different absorbent capacities.

U.S. Pat. No. 5,897,542 issued to Lash et al. on Apr. 27, 1999 is addressed to this problem and discloses absorbent articles having different absorbent capacities are stored or contained in a single package.

It is generally preferred that the absorbent articles having different absorbent capacities contained in a single package are used or consumed by the user equally. Otherwise, there can be a situation that a first type of absorbent articles having a first absorbent capacity are consumed completely, while a second type of absorbent articles having a second absorbent capacity (which is different from the first absorbent capacity) are left in the package. In that event, the user needs to purchase the first type of absorbent articles although the second type of the absorbent articles still remains in the package.

This issue is more problematic when a plurality of types of feminine hygiene articles (e.g., sanitary napkins, tampons, interlabial products, incontinence devices, and pantiliners) having different absorbent capacities are contained in a single package. It is believed that such a design (i.e., a plurality of types of feminine hygiene articles are contained in a single package) is preferred to provide a better protection from soiling during one menstruation period. It is generally known that the flow level of discharged body fluids per day changes depending on the days during the menstruation period. To provide a desired protection from soiling, it is preferred that a plurality of types of feminine hygiene articles having different absorbent capacities are used appropriately. Thus, a single package product which contains a plurality of types of feminine hygiene articles is preferred. Such feminine hygiene articles are expected to be used or consumed equally. Otherwise, the single package product which is designed to be used or consumed for one menstruation period is not enough to provide an expected protection from soiling.

Based on the foregoing, there is a need for a disposable absorbent product which includes a plurality of types of disposable absorbent articles contained in a single package that can help users to consume the contained disposable absorbent articles equally. There is also a need for a disposable absorbent product which includes a plurality of types of feminine hygiene articles contained in a single package that can help users to consume the contained feminine hygiene articles equally to provide a better protection during the menstruation period.

### SUMMARY OF THE INVENTION

The present invention is directed to a disposable absorbent product, comprising: a package having at least one window and an outer surface; and a plurality of disposable absorbent articles contained in the package. The plurality of disposable absorbent articles include absorbent articles wherein each type of absorbent articles is identified by an indication means disposed on the respective absorbent articles. The indication means of the absorbent articles can be seen through the at least one window.

The foregoing answers the need for a disposable absorbent product which includes a plurality of types of disposable absorbent articles contained in a single package that can help users to consume the contained disposable absorbent articles equally.

These and other features, aspects, and advantages of the present invention will become evident to those skilled in the art from reading of the present disclosure.

### BRIEF DESCRIPTION OF THE DRAWING

While the specification concludes with claims particularly pointing out and distinctly claiming the invention, it is believed that the invention will be better understood from the following description of preferred embodiments taken in conjunction with the accompanying drawings wherein like designations are used to designate substantially identical elements, and in which:

Figure is a simplified perspective illustration of a disposable absorbent product which is one preferred embodiment of the present invention.

### DETAILED DESCRIPTION OF THE INVENTION

All cited references are incorporated herein by reference in their entirety. Citation of any reference is not an admission regarding any determination as to its availability as prior art to the claimed invention.

Herein, “comprise”, “include” and “contain” mean that other element(s) and step(s) which do not affect the end result can be added. These terms encompass the terms “consisting of” and “consisting essentially of”.

Herein, “joined” or “joining” encompasses configurations whereby an element is directly secured to another by affixing the element directly to the other element, and configurations whereby the element is indirectly secured to the other element by affixing the element to intermediate member(s) which in turn are affixed to the other element.

Herein, the term “absorbent article” refers to devices which absorb and contain body exudates, and more specifically, refers to devices which are placed against or in proximity to the body of the wearer to absorb and contain the various body fluids or exudates discharged from the body. The term “disposable” is used herein to describe absorbent articles which are not intended to be laundered or otherwise restored or reused as an absorbent article (i.e., they are intended to be discarded after a single use and, preferably, to be recycled, composted or otherwise disposed of in an environmentally compatible manner). Disposable absorbent articles include feminine hygiene articles, diapers, incontinent briefs, incontinent undergarments, incontinent pads, training pants, and the like. A preferred disposable absorbent article of the present invention is a feminine hygiene article.

Herein, the term “feminine hygiene articles” refer to a disposable absorbent article used by women for catamenial protection. Such feminine hygiene articles include sanitary napkins, tampons, interlabial products, incontinence devices, and pantiliners.

Herein, the term “different types of absorbent articles” refers to absorbent articles which include at least one of the absorbent articles having a different physical property or structure from that of the other one of the absorbent articles. Examples of physical property or structure include the absorbent capacity of absorbent articles, the dimension(s) (e.g., the longitudinal length, the traversal width and/or the thickness) of absorbent articles, the kinds of absorbent articles (e.g., sanitary napkins, tampons, interlabial products, incontinence devices, pantiliners, diapers, incontinent briefs, incontinent undergarments, and training pants.), and the combination thereof.

Figure is a simplified perspective illustration of a disposable absorbent product 100 which is one preferred embodiment of the present invention. The disposable absorbent product 100 includes a package 10 which contains a plurality of disposable absorbent articles 20 having at least two different types of absorbent articles. These disposable absorbent articles 20 are stacked to form at least one stack 22 in the package 10.

The package 10 can be formed by any material and can take any structure known in the art. For example, the package 10 may be a carton which is formed by a cardboard material. In the preferred embodiment shown in Figure, the package 10 is a flexible bag which is formed by a thin film material. Such a thin film material may be made of paper, plastic, or any recyclable material, and may take a laminate structure comprised of two or more of the aforementioned materials. The package material may also be of non-biodegradable or non-recyclable materials, such as polymeric films (e.g., polypropylene films and polyethylene films).

The package 10 shown in Figure is a flexible bag which is preferably formed from a continuous tube of a thin film material (e.g., a polyethylene film) having an axis parallel to the height of the package 10. The top and bottom portions of the package 10 are closed by forming side gussets 26. A preferred film material for the package 10 of the embodiment shown in Figure is a polyethylene film. The package 10 includes a front panel 11, a rear panel 12 opposed to the front panel 11, side panels 13 which connects the front and rear panels 11 and 12, a top panel 14 which connects the front, rear, and side panels 11, 12 and 13, and a bottom panel 15 opposed to the top panel 14. Each pair of the front and rear panels 11 and 12, the side panels 13 and the top and bottom panels 14 and 15 are substantially planar as shown in Figure. These panels 11-15 form an outer surface 24 of the package 10.

The disposable absorbent articles 20 contained in the package 10 include at least two different types of absorbent articles. Preferably, the disposable absorbent articles 20 contained in the package 10 are feminine hygiene articles such as sanitary napkins. In the embodiment shown in Figure, three types of sanitary napkins 30 are contained as the disposable absorbent articles 20. Such three types of sanitary napkins 30 have different dimensions so that they have different absorbent capacities. In a preferred embodiment, a first type of the sanitary napkins 30 has a longitudinal length of about 320 mm (for the highest absorbent capacity for the overnight use or a heavy flow use), a second type of the sanitary napkins 30 has a longitudinal length of about 280 mm (for the middle absorbent capacity for a moderate flow use), and a third type of the sanitary napkins 30 has a longitudinal length of about 240 mm (for the lowest absorbent capacity for the light daytime use or a low flow use). These changes in the longitudinal lengths of the sanitary napkins 30 can provide differences in the absorbent capacities.

Such differences in the absorbent capacities are preferred by users since the disposable absorbent articles 20 which are consumed in one day by a user normally have different usage time lengths. For example, the diapers which are used in the daytime are replaced more often than the diapers which are used in the nighttime. Similarly, depending on the days in the menstruation period, the amount of body fluids discharged from the body of a woman generally changes. For example, the sanitary napkins which are used in the second day of the menstruation period are replaced more often than the sanitary napkins which are used in the other days in the menstruation period. From these facts, appropriately selecting disposable absorbent articles which have different absorbent capacities is preferred by users.

In a preferred embodiment, each of the disposable absorbent articles 20 includes an individual flexible wrapper or bag (not shown in Figure) which wraps or contains the respective absorbent article 20. When each of the disposable absorbent articles 20 is wrapped by an individual flexible wrapper or bag, the outer surface of the absorbent article is a part of the wrapper or bag which faces the outside of the article. Alternatively, the disposable absorbent articles 20 can be just stored in the package 10 without being individually wrapped or contained by a flexible wrapper or bag.

The disposable absorbent articles 20 can be stored in the package 10 without being folded. Preferably, at least a part of the disposable absorbent articles 20 are folded inwardly. The folding operation for the disposable absorbent articles 20 is preferably carried out before or when they are stored in the package 10 or the flexible wrapper or bag. For example, when the disposable absorbent articles 20 are sanitary napkins, each sanitary napkin is folded along at least one folding line, preferably two folding lines so that the sanitary napkin is folded into at least two (or three) sections which are defined by the folding line(s). Preferred manners for folding and/or wrapping feminine hygiene articles (e.g., sanitary napkins) are disclosed in U.S. Pat. No. 6,074,376 issued to Mills on Jun. 13, 2000 and U.S. Pat. No. 5,569,228 issued to Byrd et al. on Oct. 29, 1996.

Each type of absorbent articles is visually identified by a common indication means which is disposed on the outer surface of the respective absorbent articles. Herein, “outer surface” of an absorbent article refers to a part of the absorbent article which faces the outside of the article. Any indication means known in the art can be used as long as it can visually differentiate one type of absorbent articles from the other.

The indication means can be any of colors, lines, patterns, ornamental designs, pictures, symbols, characters (or codes), and the combination thereof, or other markings which can be disposed on the disposable absorbent articles 20. The indication means identifies each type of absorbent articles 20 contained in the package 10. In the embodiment shown in Figure, if each of the disposable absorbent articles 20 is individually wrapped or contained in a wrapper or bag, the indication means is disposed on the wrapper or bag. In a preferred embodiment, the indication means is a color variation made in the respective wrapper films for corresponding types of the absorbent articles.

Alternatively, if the disposable absorbent articles 20 are not individually wrapped or contained in a wrapper or bag (i.e., they are merely directly stored in the package 10), the indication means is disposed on at least one component materials (e.g., the backsheet, the topsheet, etc.) of each disposable absorbent article 20 which can seen. In a preferred embodiment, the indication means is a color variation made in the respective backsheet materials for corresponding types of the absorbent articles.

In the embodiment shown in Figure, the stack 22 is provided so that the side panels 13 of the package 10 are substantially equal to the cross-section of the stack 22 of absorbent articles 20. The stack 22 includes at least two types of disposable absorbent articles 20. In the embodiment shown in Figure, three types of sanitary napkins 30 are included in the stack 22.

The total number of the disposable absorbent articles 20 contained in the package 10 may change depending on the target usage time length for one package product. For example, in the embodiment wherein the disposable absorbent articles 20 are sanitary napkins, the total number of the sanitary napkins 30 is preferably from 15 to 25. Preferably, the sanitary napkins 30 for the overnight use or a heavy flow use is from 6 to 12, the sanitary napkins 30 for a moderate flow use is from 4 to 8, and the sanitary napkins 30 for the light daytime use or a low flow use is from 3 to 7.

In an alternative preferred embodiment wherein the disposable absorbent articles 20 are sanitary napkins and the target usage time length for one package is one menstruation period, i.e., all the sanitary napkins contained in the package 10 are expected to be consumed about within one menstruation period, the total number of the sanitary napkins is preferably from 15 to 22. Preferably, the sanitary napkins for the overnight use or a heavy flow use is from 6 to 12, the sanitary napkins 30 for a moderate flow use is from 4 to 8, and the sanitary napkins 30 for the light daytime use or a low flow use is from 3 to 5.

When the disposable absorbent articles 20 contained in the one package 10 are other kinds of absorbent articles than the sanitary napkins such as tampons, interlabial products, incontinence devices, pantiliners, diapers, incontinent briefs, incontinent undergarments, and training pants, the total number as well as the number of each type of the disposable absorbent articles 20 contained in one package is determined by considering the absorbent capacities and the recommendable usage time lengths of such disposable absorbent articles 20.

The package 10 has at least one window 16. The indication means which is disposed on the absorbent articles 20 is seen through the window 16. The window 16 can be provided at any panels of the package 10. If desired, a plurality of the windows may be provided in the package 10. If desired, additional window(s) may be provided in, for example, the rear panel 12 or the side panels 13. The window 16 can take any shape such as a circle, a square, a rectangle, a trapezoid, an ellipse, a triangle or any other shape such as shown in Figure. The window 16 can have any sizes or dimensions as long as the indication means disposed on the absorbent articles 20 can be seen through the window 16. In a preferred embodiment, the window 16 has an enough dimension (e.g., the length and the width for a rectangular window) so that at least two types, more preferably all types of the contained absorbent articles can be seen through the window 16. Preferably, the window 16 has an enough size so that at least 30%, more preferably about from 60% to about 100% of the contained absorbent articles can be seen through the window.

Since the disposable absorbent articles 20 contained in the package 10 can be seen by users through the window 16, users can consume the plurality of types of disposable absorbent articles 20 equally. The window 16 also enables consumers (or purchasers) to understand the plurality of types of disposable absorbent articles 20 are contained within the package 10 at the point of purchase.

The window 16 should include a portion which is at least translucent, and more preferably transparent. The window 16 does not necessarily need to be completely transparent. The window 16 may be somewhat translucent as long as the indication means which are disposed on the absorbent articles 20 can be seen through the window 16.

The window 16 can be formed by any means known in the art. Preferably, the window 16 is formed by differentiating the translucency or the transparency at the window 16 from the area surrounding the window 16. Such a differentiation can be made by using different paints between the area within the window 16 and the area surrounding the window 16. In the embodiment shown in Figure, the window 16 can be formed by applying no paint in the area of the window 16 so that it has a very high transparency, while applying a paint which makes the surrounding area of the window 16 translucent or non-transparent. The rest of the outer surface 24 of the package 10 may have one or more colors as well as lines, patterns, ornamental designs, pictures, symbols, characters (or codes), and the combination thereof.

In the embodiment wherein the package 10 is formed by a cardboard material, the window 16 is formed by removing at least one part of the cardboard material by cutting. Preferably, the cut portion is covered by a separate sheet material (e.g., a translucent or transparent film material) which is attached to the inside surface of the package wall at the window portion so that the contained absorbent articles 20 can be effectively sealed or protected from the outside air.

Preferably, the outer surface 24 of the package 10 provides first information, while the window 16 provides second information which has a relevancy to the first information. The first and second information can be any information as long as they can have a relevancy. The first information can be indicated in any panel of the package 10. The first information is preferably indicated in the panel where the window 16 is provided.

In one preferred embodiment, the window 16 has a shape (i.e., the second information) which has a relevancy to the first information. For example, in the embodiment shown in Figure, the window 16 of the front panel 11 has a shape that is similar to the flow level change during one menstruation period (i.e., the second information), while the front panel 11 also has a graph 17 which shows the same or relevant information (i.e., the first information) near the window 16, i.e., the graph 17 shows the flow level (FL) changes depending on the days (DAY) during one menstruation period. Herein, the term “flow level” refers to the total amount of body fluids discharged per one day from a woman.

In another preferred embodiment, the window 16 shows the stacked sanitary napkins 30 having a plurality of types wherein the respective types are identified by the indication means, for example, different colors (i.e., the second information). The same or similar colors are also used in the graph 17 to provide the first information. For example, the graph 17 shows the flow level changes per day by a plurality of bars during one menstruation period as shown in Figure. The bars are indicated by different colors which are also used in the indication means disposed on the sanitary napkins 30. By using the same (or similar) colors for the indication means on the sanitary napkins 30 and the graph 17, the user can easily notice what sanitary napkins are recommended to use in each day in the menstruation period.

An opening device 18 is preferably provided in the top panel 14. The opening device 18 preferably has an enough size (e.g., the length) so that different types of absorbent articles 20 can be picked up by the user. The opening device 18 can take any shape and dimension as long as different types of absorbent articles 20 contained in the package 10 can be picked up by the user. The opening device 18 helps users to consume the plurality of types of disposable absorbent articles 20 equally.

The opening device 18 preferably includes a line of weakness 19 which extends within the top panel 14. The line of weakness 19 may extend to other panels than the top panel 14 such as the side panels 13, the front panel 11 and/or the rear panel 12.

The opening device 18 includes a substantially continuous line of weakness which traverses the top panel 14 in an area generally coinciding with the length of the stack 22 of disposable absorbent articles 20. In the embodiment shown in Figure, the substantially continuous line of weakness 19 includes a line of perforation formed in the top panel 14. Since the continuous line of weakness 19 is located in the top panel 14 which is substantially untensioned (or low tensioned), this line of weakness 19 can be designed to rupture at relatively low levels of applied force.

It is understood that the examples and embodiments described herein are for illustrative purpose only and that various modifications or changes will be suggested to one skilled in the art without departing from the scope of the present invention.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A disposable absorbent product, comprising:
a package having at least one window and an outer surface; and
a plurality of disposable absorbent articles contained in the package, the plurality of disposable absorbent articles including one type of absorbent article wherein said type of absorbent article has a predetermined absorbency which is identified by an indication means disposed on the respective absorbent article, said indication means comprising a color wherein said color indicates the predetermined absorbency,
wherein the indication means of said absorbent articles can be seen through the at least one window.

2. The disposable absorbent product of claim 1, wherein the one type of absorbent article are defined by absorbent capacity, the kind, the dimension, or the combination thereof.

3. The disposable absorbent product of claim 1, wherein the window has enough size so that at least 30% of the contained absorbent articles can be seen through the window.

4. The disposable absorbent product of claim 1, wherein the indication means includes colors, lines, patterns, ornamental designs, pictures, symbols, script, characters, and the combination thereof.

5. The disposable absorbent product of claim 1, wherein the package has a front panel, a rear panels opposed to the front panel, side panels which connects the front and rear panels, and a top panel which connects the front, rear, and side panels, and wherein the window is formed on the front panel and an opening structure is formed on the top panel.

6. The disposable absorbent product of claim 1, wherein the window is formed by differentiating the translucency or the transparency at the window from the area surrounding the window.

7. The disposable absorbent product of claim 1, wherein each of the disposable absorbent articles includes an individual flexible wrapper which wraps or contains the respective absorbent article, the wrapper comprises a wrapper indication means comprising a color, the wrapper indication means being a same similar color to said color of said indication means for said absorbent article.

8. The disposable absorbent product of claim 1, wherein the outer surface of the package provides first information, while the window provides second information which has a relevancy to the first information.

9. The disposable absorbent product of claim 1, wherein the disposable absorbent articles are feminine hygiene articles.

10. A disposable absorbent product, comprising:
a package having at least one window and an outer surface; and
a plurality of disposable absorbent articles contained in the package, the plurality of disposable absorbent articles including one type of absorbent article wherein said type of absorbent article is identified by an absorbent article indication means,
wherein the absorbent article indication means is a color which can be seen through the at least one window, and wherein the package comprises a package indication means, the package indication means being a same or similar color.

11. The disposable absorbent product of claim 10, wherein the absorbent articles are defined by the absorbent capacity, the kind, the dimension, or the combination thereof.

12. The disposable absorbent product of claim 10, wherein the window has enough size so that at least 30% of the contained absorbent articles can be seen through the window.

13. The disposable absorbent product of claim 10, wherein the indication means includes colors, lines, patterns, ornamental designs, pictures, symbols, script, characters and the combination thereof.

14. A packaged disposable absorbent product, comprising:
a package having at least one window and an outer surface; and
a plurality of feminine hygiene articles contained in the package, the plurality of feminine hygiene articles including one type of feminine hygiene article wherein each of the one type of feminine hygiene articles is wrapped or contained in an individual flexible wrapper, and wherein an absorbent article indication means is disposed on the wrapper, said absorbent article indication means being a color, wherein said color indicates a predetermined absorbency,
wherein the indication means of said wrapper can be seen through the at least one window.

15. The packaged disposable absorbent product of claim 14, wherein the package comprises a package indication means wherein said package indication means comprises a color, the package indication means being a same or similar color to the absorbent article indication means.

16. The packaged disposable absorbent product of claim 14, wherein the one type feminine hygiene articles are defined by absorbent capacity, the kind, the dimension, or the combination thereof.

17. The packaged disposable absorbent product of claim 14, wherein the window has enough size so that at least 30% of the contained feminine hygiene articles can be seen through the window.

18. The packaged disposable absorbent product of claim 14, wherein the indication means includes colors, lines, patterns, ornamental designs, pictures, symbols, script, characters and the combination thereof.
